(19) United States
(12) Patent Application Publication
(10) Pub. No.: US 2026/0174483 A1
(43) Pub. Date: Jun. 25, 2026

(54) OSTEOSYNTHESIS PLATE

(71) Applicant: Karl Leibinger Asset Management GmbH & Co. KG, Mühlheim an der Donau (DE)

(72) Inventors: Ralf Leibinger, Mühlheim (DE); Frank Reinauer, Emmingen-Liptingen (DE); Steffen Aberle, Königsfeld (DE)

(21) Appl. No.: 19/424,911

(22) Filed: Dec. 18, 2025

(30) Foreign Application Priority Data
Dec. 19, 2024 (DE) 102024138940.0

(51) Int. Cl. A61B 17/80

(52) U.S. Cl. CPC A61B 17/8071

(57) ABSTRACT

An osteosynthesis plate (OP; OP′; OP″; OP′″) for fixation of bone fragments (K1, K2) for restoration of a bone (K) includes a plate body (P; P′) having a top side (OS) and an underside with which the plate body (P; P′) is placeable on the bone fragments (K1, K2). The plate body (P; P′) has a plurality of fixation sections (BA1, BA2; BA1′, BA2′, BA3′) at which, on the underside thereof, fixations of the osteosynthesis plate (OP; OP′; OP″; OP′″) to the bone fragments (K1, K2) is implementable and with each of which at least one fixation point (BP1, BP2; BP1′, BP2′, BP3′) is associated. The at least one fixation point (BP2; BP2′, BP3′) on at least one of the fixation sections (BA1, BA2; BA1′, BA2′, BA3′) is formed on an island section (IA) connected to the associated fixation section (BA2; BA2′, BA3′) of the plate body (P; P′) exclusively via spring segments (FS1, FS2) to permit relative displacement of the respective fixation section (BA2; BA2′, BA3′) and of the respective island section (IA) in an elastically resilient manner. The spring segments (FS1, FS2) asymmetrically surround the fixation point (BP2, BP2′, BP3′) associated therewith.

### CROSS-REFERENCE TO RELATED APPLICATION

The present application is related and has right of priority to German Patent Application No. DE 102024138940.0 filed on Dec. 19, 2025, which is incorporated by reference in its entirety for all purposes.

### TECHNICAL FIELD

The invention relates generally to an osteosynthesis plate provided for a fixation of bone fragments for the restoration of a bone, in particular a mandible

### BACKGROUND

In osteosynthesis, two or more bone fragments are connected to one another within the framework of a surgical procedure in order to enable the bone fragments to grow together and thus achieve a restoration of the bone. The goal of osteosynthesis is to stabilize the bone fragments with respect to one another, wherein this stabilization is to take place in a correct position and thus, optionally, while correcting malalignments. In addition to a fixation using wire or screws, osteosynthesis plates are also used depending on the area of application, wherein the respective osteosynthesis plate is placed on the respective bone in the region of a respective gap between the bone fragments and fixated to each of the bone fragments to be connected to one another.

In addition to stabilizing bone fragments after a bone fracture, osteosynthesis plates are also used in part when a bone segment resected elsewhere is to be used at an existing defect in a bone and stabilized for a restoration of the bone. Such a defect can arise, for example, due to the need to remove a section of bone after a tumor disease.

With respect to a healing process of the bone, it has proven advantageous when the bone fragments of the bone to be restored are not rigidly connected to one another via the osteosynthesis plate, but rather when micromotions of the bone fragments relative to one another are made possible. In order to enable these micromotions, the fixation points are connected via spring segments to at least one of the bone fragments to be connected.

WO 2011/163387 A2 describes a plate for fixation of a bone fracture having an outer surface and a surface facing the bone. The bone plate has slots which extend through the plate from the outer surface to the surface facing the bone. The slots at least partially circumscribe a periphery of one or a plurality of receiving holes and do not extend through a longitudinal edge of the bone plate. The slots form a spring element which at least partially surrounds the receiving hole and permits an axial displacement of the bone plate relative to the one receiving hole or the plurality of receiving holes within a plane which is parallel to an upper surface or a lower surface of the bone plate but prevents a movement of the bone plate relative to the one receiving hole or the plurality of receiving holes in a direction that is perpendicular to the top side of the bone plate.

### BRIEF SUMMARY

Example aspects of the present invention provide an osteosynthesis plate with which an arrangement of fixation points that is as variable as possible can be provided, which fixation points are displaceable in a relative manner via spring segments.

According to example aspects of the invention, an osteosynthesis plate, which is provided for a fixation of bone fragments for the restoration of a bone, includes a plate body having a top side and an underside with which the plate body is to be placed on the bone fragments. The plate body has a plurality of fixation sections at which, on the underside thereof, the osteosynthesis plate is to be fixated to the bone fragments and with each of which at least one fixation point is associated for this purpose. The at least one fixation point, on at least one of the fixation sections, is formed on an island section which is connected to the associated fixation section of the plate body exclusively via spring segments which permit a relative displacement of the respective fixation section and of the respective island section relative to one another in an elastically resilient manner.

The osteosynthesis plate according to example aspects of the invention is provided for a fixation of bone fragments for the restoration of a bone. This restoration of the bone can be present within the framework of the invention in such a way that bone fragments that have formed due to a fracture of the bone are fixated to one another via the osteosynthesis plate according to example aspects of the invention. In this case, the bone fragments associated with the bone to be restored are therefore fixated and stabilized with respect to one another via the osteosynthesis plate. A restoration of a bone can also be considered within the meaning of the invention to mean, however, that a defect of a bone is closed with a bone segment which has been resected from another bone for this purpose. Accordingly, the bone in this case is restored from its own bone fragments and a bone fragment associated with another bone. The osteosynthesis plate according to example aspects of the invention is then used in this case for the fixation of the resected bone segment closing the defect with at least one of the bone segments located on each side. Most particularly preferably, the osteosynthesis plate is provided for use in the region of the lower jaw bone (mandible), wherein a restoration can then be achieved either within the framework of a bone fracture or within the framework of closing a bone defect.

The osteosynthesis plate is equipped with a plate body, which is preferably elongate, i.e. extends in particular at least predominantly in a longitudinal direction. The plate body can extend along a longitudinal axis extending in this longitudinal direction or can be designed extending along a trajectory oriented in this longitudinal direction.

The plate body has a top side and an underside, which are oriented, in particular, facing away from one another on the plate body. When the osteosynthesis plate according to example aspects of the invention is used, the plate body is placed on the bone fragments with the underside of the plate body, wherein, during this placement, at least one gap is bridged, which gap extends between the bone fragments to be fixated. In addition, when the osteosynthesis plate is used, a fixation to the bone fragments to be fixated is implemented on the underside of the plate body, for the purpose of which the plate body of the osteosynthesis plate is equipped with a plurality of fixation sections. Each of the fixation sections has associated therewith at least one fixation point, wherein the respective fixation point is preferably formed by a respective through-hole, through which a bone screw can be guided. The fixation to the respective bone fragment is implemented by the bone screw.

On at least one of the fixation sections of the plate body, the at least one provided fixation point is formed on an island section, which is connected to the associated fixation section exclusively via spring segments, which couple the respective island section to the associated fixation section. These spring segments permit a relative displacement of the island section relative to the associated fixation section in an elastically resilient manner, which means, within the meaning of example aspects of the invention, in particular that the spring segment elastically deforms during a relative displacement that the island section and the associated fixation section undergo relative to one another when loaded and, when unloaded, returns to its original shape while moving back into an initial position. Preferably, the spring segment is designed as a spring leg extending at least largely linearly.

Preferably, the spring segments permit the relative displacement between island section and fixation section only largely parallel to the top side and the underside and/or in the longitudinal direction. This is the case because, since the respective gap between the bone fragments is also bridged in this direction due to the course of the plate body, relative displacements oriented in this direction result in changes in the gap size, which has proven advantageous with respect to the healing process of the bone to be restored.

Particularly preferably, the respective island section protrudes from the underside of the plate body relative to the associated fixation section, whereby, when the osteosynthesis plate is fixated to the respective bone fragment, the respective island section rests on the bone fragment and, at the same time, there is open space between the bone fragment and the associated fixation section. As a result, it can be ensured that the relative displacements between island section and fixation section are not impeded by friction due to the fixation section also resting on the bone fragment which is also to be displaced relative to the fixation section.

The osteosynthesis plate is preferably manufactured by an additive manufacturing process, preferably by a 3D-printing process. The osteosynthesis plate is preferably formed of titanium, a titanium alloy, of stainless steel, or of a suitable polymer, for example polyether ether ketone (PEEK).

Example aspects of the invention provide spring segments that asymmetrically surround the fixation point associated therewith.

Such an example embodiment of an osteosynthesis plate has the advantage that, due to the asymmetrical courses of the spring segments, a compact example embodiment of the island section on the associated fixation section is possible. With an island section that is arranged, on the associated fixation section, at one end of the plate body, the spring segment located at this end can be configured differently from the opposite spring segment in order to be able to arrange the island section as close as possible to the end of the plate body. If the fixation section is provided with a plurality of fixation points formed on island sections, the asymmetrical courses of the respective spring segments enable a denser arrangement of the island sections. The design according to example aspects of the invention thus enables a greater variability in the arrangement of the fixation points, such that the arrangement can be adapted to the anatomy in the best way possible.

According to one possible example embodiment of the invention, the island section is connected to the respective fixation section via exactly two respective spring segments. In this case, exactly two spring segments therefore couple the respective island section to the associated fixation section. Within the framework of example aspects of the invention, more than two spring segments could also be provided for connecting the respective island section to the associated fixation section.

According to a further possible example embodiment of the invention, the island section and the associated spring segments are formed by through-openings formed on the respective fixation section. Advantageously, this enables a one-piece design of the plate body to be obtained by defining the island section and the spring segments by introducing through-openings in the plate body at the respective fixation section. If the plate body of the osteosynthesis plate is manufactured by an additive manufacturing process, the through-openings may also have been formed in this additive manufacturing process. Alternatively, the through-openings can also be defined in an abrasive or material-removing manner, however.

The through-openings can be U-shaped, wherein the U-shaped through-openings are nested inside one another with their openings facing one another. As a result, the respective island section and the associated spring segments can be designed to be particularly space-saving, such that the variability of the arrangement of the fixation points is further improved.

In a further possible example embodiment of the invention, a first one of the spring segments is oriented orthogonally to a longitudinal direction of the fixation section. Due to this course of the spring segment, the associated island section can be arranged close to one end of the associated fixation section or adjacent to a further island section of the associated fixation section.

According to a further possible example embodiment of the invention, the respective fixation section has associated therewith a plurality of fixation points, wherein each of the fixation points is formed on a respective island section with spring segments arranged asymmetrically around the respective fixation point. As a result, the plurality of fixation points on this fixation section can be arranged in a compact manner relative to one another, such that the variability of the arrangement of the fixation points is further improved.

Preferably, a second one of the spring segments is aligned extending at an acute angle to a direction which is orthogonal to a longitudinal direction of the fixation section. As a result, a relative displacement of the island section relative to the associated fixation section can be brought about and thus a change in the gap between the bone fragments is effected via this spring segment, which is aligned at an acute angle, when a transverse load is applied to the fixation section or when a load extending in a transverse direction is introduced at the fixation point.

In a combination of the two above-described example variants, the plurality of fixation points formed on island sections can be consecutively formed in the longitudinal direction of the respective fixation section, wherein adjacent island sections are designed identically to one another and arranged in a point-mirrored manner, in that the adjacent island sections face one another with the spring segments extending at an acute angle. Advantageously, the consecutive island sections can thereby be formed extremely compactly next to one another.

In a further possible example embodiment of the invention, the fixation sections are located in pairs on each side of a respective intermediate bridge section which is provided for bridging a gap extending between the bone fragments. In a development of this possible example embodiment, at least one of the asymmetrically arranged spring segments can be aligned in such a way that a relative displacement between the respective island section and the respective fixation section takes place when a load is applied in a main loading direction of the bone, during which relative displacement a distance between the respective island section and the bridge section increases. This has the advantage that, when a load is applied to the bone in the main loading direction, the gap bridged by the bridge section between the bone segments also enlarges due to the increase in the distance between the island section and the bridge section. This prevents the gap between the bone segments from becoming zero and the bone segments impacting one another, which would otherwise complicate the healing process of the bone to be restored. When a resected bone segment is inserted into a defect of the bone to be restored, a gap between the bone segments that is too small or non-existent can also thereby be prevented when the resected bone segment is configured with a transition fit with respect to the defect.

Within the meaning of the invention, “loaded in the main loading direction” of the bone is understood to mean, in particular, an action of force on the bone when same is loaded as usual. Preferably, this loading takes place in such a way that a resulting transverse force is induced on the associated fixation point on the island section. In the case of the preferred example embodiment of the osteosynthesis plate for the fixation of bone fragments for the restoration of a mandible, the load in the main loading direction is, in particular, a biting force to be supported via the mandible.

According to a further possible example embodiment of the invention, the at least one fixation point on one of the fixation sections is fixed in place. On this fixation section, the one fixation point or the multiple of fixation points is/are fixed in place, such that rigid connections to the respective bone segment can be established.

Alternatively or additionally, the fixation points of a plurality of fixation sections are formed on island sections. If a positionally fixed design of the at least one fixation point has not been implemented on any of the fixation sections, the fixation points of all fixation sections are formed on island sections.

### BRIEF DESCRIPTION OF THE DRAWINGS

Advantageous embodiments of the invention, which are explained in the following, are shown in the drawings. In the drawings:

FIGS. 1A and 1B show schematic representations of a bone with an osteosynthesis plate according to one example embodiment of the invention fixated thereto, shown in different states; and

FIGS. 2 through 4 show perspective views of a respective osteosynthesis plate according to a respective further example embodiment of the invention.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

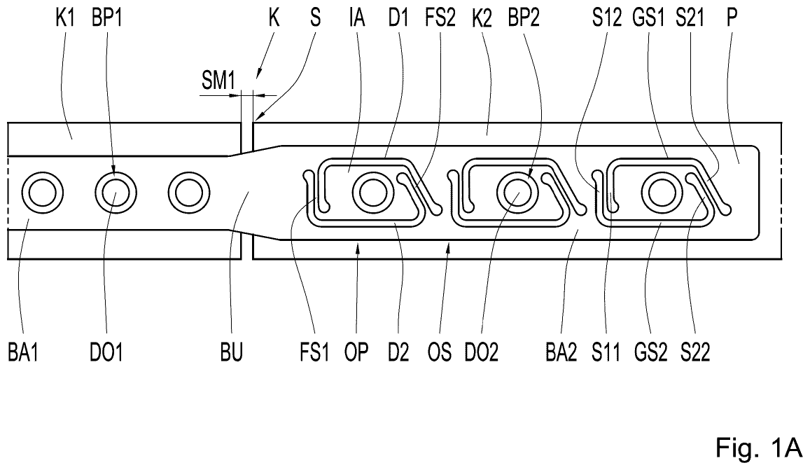

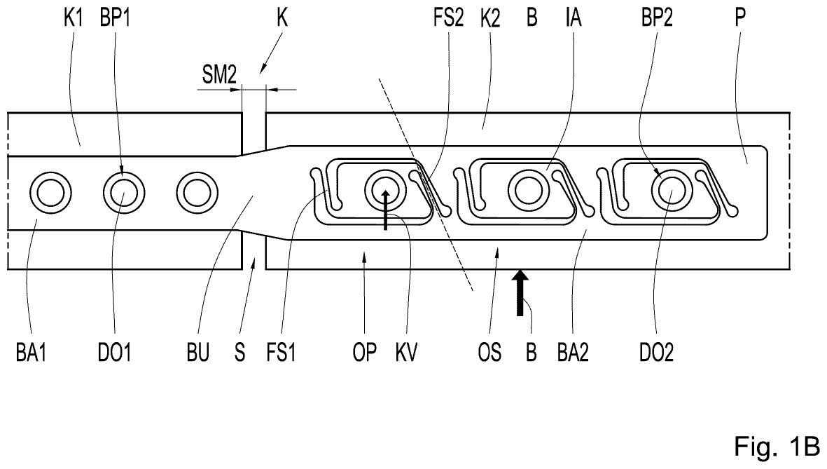

FIGS. 1A and 1B show schematic representations of a bone K with an osteosynthesis plate OP fixated thereto, wherein the bone K is preferably a mandible in this case. The osteosynthesis plate OP is to be fixated to bone fragments K1 and K2 in order to stabilize the bone fragments K1 and K2 relative to one another and thereby enable the bone fragments K1 and K2 to grow together for the restoration of the bone K.

In the present case, the bone fragments K1 and K2 may have formed due to a fracture of the bone K, wherein the osteosynthesis plate OP is then provided for healing this bone fracture by stabilizing the bone fragments K1 and K2 relative to one another. Alternatively, one of the bone fragments K1 and K2 may have been used to close a defect of the bone K, for example, because a part of the bone K had to be removed at this point due to a tumor disease. The bone fragment K1 or K2 is a section of bone resected from another bone, for example, from the fibula.

The osteosynthesis plate OP has an elongate plate body P, which preferably consists of titanium or a titanium alloy and has been produced, in particular, in an additive manufacturing process, preferably in the 3D printing process. The plate body P has a top side OS, which is visible in FIGS. 1A and 1B, and a non-visible underside, which is arranged opposite thereto and with which the plate body P is placed on the bone fragments K1 and K2.

The plate body P is composed of two fixation sections BA1 and BA2 and a bridge section BU located between the fixation sections BA1 and BA2. A fixation of the plate body P to the bone fragment K1 is to be implemented at the fixation section BA1 on the underside of the plate body P, wherein this fixation is implemented at fixation points BP1, which are associated with the fixation section BA1. The fixation points BP1 are designed as through-holes DO1, each of which extends between the top side OS and the underside and is used for guiding a respective bone screw (not shown in the figures) through. The through-holes DO1 are introduced in the fixation section BA1 and fixed in place on the fixation section BA1.

Adjoining the fixation section BA1, the plate body P has a bridge section BU for bridging a gap S. The gap S is located between the bone fragments K1 and K2. Adjoining the bridge section BU is the fixation section BA2 at which the plate body P is fixated to the bone fragment K2. The fixation section BA2 has fixation points BP2 associated therewith for this purpose. The fixation points BP2 are also designed as through-holes DO2 which extend between the top side OS and the underside and are used for guiding a respective bone screw (not shown in the figures) through.

In contrast to the fixation section BA1, the through-holes DO2 in the fixation section BA2 are introduced, or defined, in island sections IA, which are separated from the fixation section BA2 by connecting each individual island section IA to the fixation section BA2 exclusively via spring segments FS1 and FS2. The associated spring segments FS1 and FS2 permit elastically resilient relative displacements of the respective island section IA, and thus also of the associated fixation point BP2, relative to the fixation section BA2.

The spring segments FS1 and FS2 and also the respective associated island section IA are defined on the osteosynthesis plate OP via through-openings D1 and D2 in the plate body P, which are U-shaped and extend from the top side OS to the underside. The through-openings D1 and D2 are nested inside one another with the openings of their U-shape facing one another, whereby in addition to separating the respective island section IA from the fixation section BA2, the spring segments FS1 and FS2 are defined. The U-shapes of the through-openings D1 and D2 deviate from one another, specifically in that both U-shapes each have a first leg S11, S12 extending transversely to a respective base side GS1, GS2 of the U-shape; in the U-shape of the through-opening D1, however, a second leg S21 extends at an angle outward relative to the base side GS1, whereas a second leg S22 in the U-shape of the through-opening D2 is angled inward. This yields linear and asymmetrical courses of the spring segments FS1 and FS2 with respect to the respective fixation point BP2. Due to these asymmetrical courses, the island sections IA provided on the fixation section BA2 can be closely spaced in the same direction.

FIG. 1A shows an unloaded state of the bone K, wherein the spring segments FS1 and FS2 of the respective island section IA set an initial position of the respective island section IA, thereby resulting in a gap size SM1 of the gap S between the bone fragments K1 and K2. In contrast, FIG. 1B shows the bone K under a load B, which is indicated in FIG. 1B with an arrow and represents a typical load on the bone K. This load B in the case of the preferred example embodiment of the bone K as a mandible can be a biting force to be supported.

The spring segment FS2 of the respective island section IA extends at an acute angle to a respective force vector KV, by which a transverse force resulting on the respective fixation point BP2 due to the load B is represented and which is indicated using an arrow in FIG. 1B for one of the fixation points BP2. In addition to this acute-angled course-indicated in FIG. 1B with a dashed line—with respect to the force vector KV, the spring segment FS2 of the island section IA extends in a divergent manner toward the bridge section BU. As a result, force is deflected at the spring segment FS2 when the load B is introduced into the bone fragment K2, causing the island sections IA to be moved away from the bridge section BU and thus increasing a distance between the island sections IA and the bridge section BU. This results in the bone fragments K1 and K2 moving apart from one another and thus the gap S increasing to a gap size SM2. When the load B is no longer applied, the gap S then decreases once again to the gap size SM1, in that the spring segments FS1 and FS2 of the respective island section IA induce a corresponding return displacement by elastically returning to their original shape. In general, the healing process of the bone K is promoted by the relative motion of the bone fragments K1 and K2 with respect to one another.

The gap sizes SM1, SM2 in FIG. 1A and FIG. 1B and the deflection of the island sections IA in FIG. 1B are shown highly exaggerated for the sake of better illustration. In an actual application of the osteosynthesis plate OP, OP′, smaller gap sizes are used, of course, and a typical load B on the bone K would result in a smaller deflection of the island sections IA than shown in FIG. 1B.

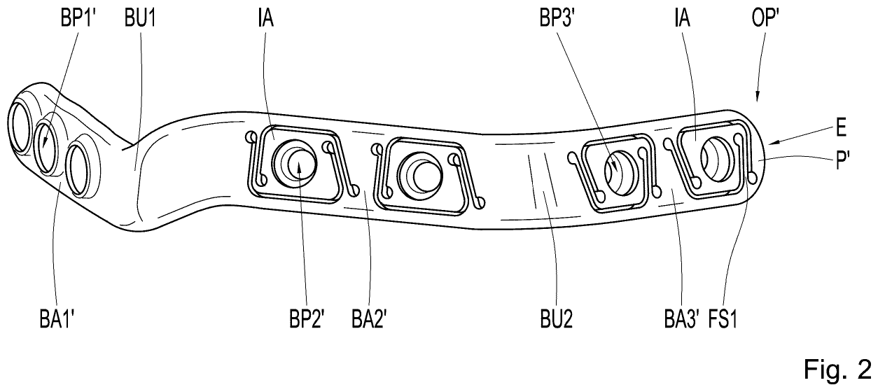

FIG. 2 shows a perspective representation of an osteosynthesis plate OP′ according to a further possible example embodiment of the invention. The difference compared to the example variant according to FIGS. 1A and 1B is that a plate body P′ of this osteosynthesis plate OP′ has two bridge sections BU1 and BU2 for bridging a respective gap (not shown in FIG. 2). Thus, three bone fragments of a bone can be stabilized with respect to one another using the osteosynthesis plate OP′ for the restoration of said bone. The bridge sections BU1 and BU2 are defined as being interposed between fixation sections BA1′, BA2′, and BA3′, each of which is used for the fixation of one of the bone fragments.

Whereas fixation points BP1′ are fixed in place on the fixation section BA1′, the fixation points BP2′ and BP3′ on the fixation sections BA2′ and BA3′ are defined on respective island sections IA′, which are designed similarly to the example variant according to FIGS. 1A and 1B. On the fixation section BA3′, the island sections IA arranged in the same direction are aligned in such a way that the spring segment FS1 of the island section IA located at one end E of the plate body P′ is located facing this end E. As a result, this final island section IA can be located close to the end E. For the rest, the possible example embodiment according to FIG. 2 corresponds to the example variant according to FIGS. 1A and 1B, such that reference is made to the descriptions thereof.

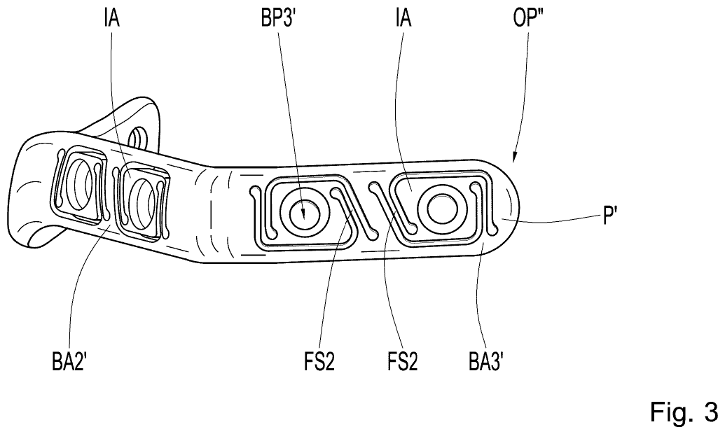

FIG. 3 shows a perspective view of an osteosynthesis plate OP″ according to a further possible example embodiment of the invention. This largely corresponds to the preceding example variant according to FIG. 2, wherein, in contrast to the osteosynthesis plate OP′ from FIG. 2, the island sections IA on both the fixation section BA2′ and on the fixation section BA3′ are oppositely directed to each other, in that, in the island sections IA oppositely directed to each other, the spring segments FS2 face one another. This enables a more compact configuration of the adjacent island sections IA on the respective fixation section BA2′ and BA3′ than is the case with the example variant according to FIG. 2. For the rest, the possible example embodiment according to FIG. 3 corresponds to the example variant according to FIG. 2, such that reference is made to the descriptions thereof.

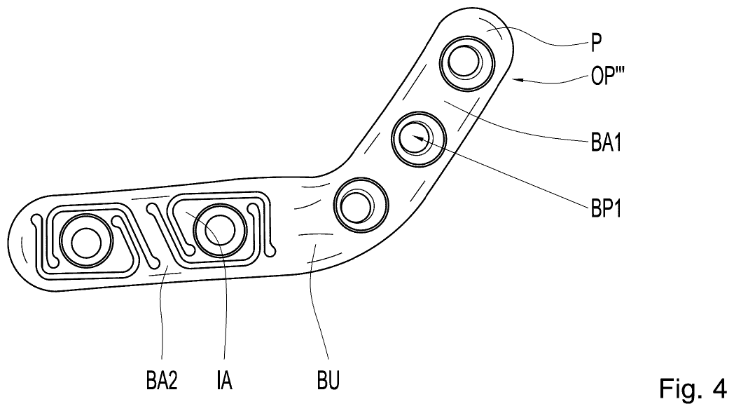

In addition, FIG. 4 shows a perspective representation of an osteosynthesis plate OP′″, which is designed corresponding to a further example embodiment of the invention and substantially corresponds to the preceding example variant according to FIG. 3. The difference is that a plate body P of this osteosynthesis plate OP′″ has only one bridge section BU and two fixation sections BA1 and BA2 located on each side thereof. Whereas fixation points BP1 on the fixation section BA1 are fixed in place, the fixation points BP2 associated with the fixation section BA2 are formed on island sections IA. In conformance with the preceding example variant according to FIG. 3, these island sections IA are oppositely directed to one another, in that the spring segments FS2 of these island sections IA face one another. For the rest, the example embodiment according to FIG. 4 corresponds to the example variant according to FIG. 3, such that reference is made to the descriptions thereof.

By the example embodiments according to example aspects of the invention, a respective osteosynthesis plate can be provided, in which a compact arrangement of fixation points is obtained, which fixation points are displaceable in a relative manner via spring segments.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

List of reference characters

| | |
|---|---|
| K | bone |
| OP, OP′, OP″, OP″′ | osteosynthesis plate |
| K1, K2 | bone fragments |
| P, P′ | plate body |
| OS | top side |
| BA1, BA2, BA1′, BA2′, BA3′ | fixation sections |
| BU, BU1, BU2 | bridge section |
| BP1, BP2, BP1′, BP2′, BP3′ | fixation points |
| DO1, DO2 | through-holes |
| S | gap |
| IA | island sections |
| FS1, FS2 | spring segments |
| D1, D2 | through-openings |
| GS1, GS2 | base sides |
| S11, S12, S21, S22 | leg |
| SM1, SM2 | gap sizes |
| B | load |
| KV | force vector |
| E | end |

1-12: (canceled)

13. An osteosynthesis plate (OP; OP′; OP″; OP′″) for a fixation of bone fragments (K1, K2) for restoration of a bone (K), comprising:
a plate body (P; P′) having a top side (OS) and an underside with which the plate body (P; P′) is placeable on the bone fragments (K1, K2), the plate body (P; P′) comprising a plurality of fixation sections (BA1, BA2; BA1′, BA2′, BA3′) at which, on the underside thereof, fixations of the osteosynthesis plate (OP; OP′; OP″; OP′″) to the bone fragments (K1, K2) is implementable and with each of which at least one fixation point (BP1, BP2; BP1′, BP2′, BP3′) is associated,
wherein the at least one fixation point (BP2; BP2′, BP3′) on at least one of the fixation sections (BA1, BA2; BA1′, BA2′, BA3′) is formed on an island section (IA) that is connected to the associated fixation section (BA2; BA2′, BA3′) of the plate body (P; P′) exclusively via spring segments (FS1, FS2) to permit relative displacement of the respective fixation section (BA2; BA2′, BA3′) and of the respective island section (IA) in an elastically resilient manner, and
wherein the spring segments (FS1, FS2) asymmetrically surround the fixation point (BP2, BP2′, BP3′) associated therewith.

14. The osteosynthesis plate (OP; OP′; OP″; OP′″) of claim 13, wherein the island section (IA) is connected to the respective fixation section (BA2, BA2′, BA3′) via exactly two spring segments (FS1, FS2).

15. The osteosynthesis plate (OP; OP′; OP″; OP′″) of claim 13, wherein the island section (IA) and the associated spring segments (FS1, FS2) are formed by through-openings (D1, D2) formed in the respective fixation section (BA2; BA2′, BA3′).

16. The osteosynthesis plate (OP; OP′; OP″; OP′″) of claim 15, wherein the through-openings (D1, D2) are U-shaped, and the U-shaped through-openings (D1, D2) are nested inside together with openings of the U-shaped through-openings (D1, D2) facing each other.

17. The osteosynthesis plate (OP; OP′; OP″; OP′″) of claim 13, wherein a first one of the spring segments (FS1, FS2) is oriented orthogonally to a longitudinal direction of the fixation section (BA2; BA2′, BA3′).

18. The osteosynthesis plate (OP; OP′; OP″; OP′″) of claim 13, wherein the respective fixation section (BA2; BA2′; BA3′) has associated therewith a plurality of fixation points (BP2; BP2′; BP3′), and each of the fixation points (BP2; BP2′; BP3′) is formed on a respective island section (IA) having spring segments (FS1, FS2) arranged asymmetrically around the respective fixation point (BP2; BP2′; BP3′).

19. The osteosynthesis plate (OP′; OP″) of claim 18, wherein the plurality of fixation points (BP2; BP2′, BP3′) formed on island sections (IA) are consecutively formed in the longitudinal direction of the respective fixation section (BA2; BA2′; BA3′), and adjacent island sections (IA) are configured identically to each other and arranged in a point-mirrored manner such that the adjacent island sections (IA) face each other with the spring segments (FS2) extending at an acute angle.

20. The osteosynthesis plate (OP; OP′; OP″; OP′″) of claim 13, wherein a second one of the spring segments (FS1, FS2) is oriented extending at an acute angle to a direction that is orthogonal to a longitudinal direction of the fixation section (BA2; BA2′, BA3′).

21. The osteosynthesis plate (OP; OP′; OP″; OP′″) of claim 13, wherein the fixation sections (BA1, BA2; BA1′, BA2′, BA3′) are located in pairs on each side of a respective intermediate bridge section (BU; BU1, BU2) that is configured for bridging a gap(S) between the bone fragments (K1, K2).

22. The osteosynthesis plate (OP; OP′) of claim 21, wherein at least one of the asymmetrically arranged spring segments (FS1, FS2) is aligned such that relative displacement between the respective island section (IA) and the respective fixation section (BA2; BA2′) takes place when a load (B) is applied in a main loading direction of the bone (K) during which a distance between the respective island section (IA) and the bridge section (BU; BU1) increases.

23. The osteosynthesis plate (OP; OP′; OP″; OP′″) of claim 13, wherein the at least one fixation point (BP1; BP1′) on one of the fixation sections (BA1, BA2; BA1′, BA2′, BA3′) is fixed.

24. The osteosynthesis plate (OP′; OP″) of claim 13, wherein the fixation points (BP2′, BP3′) of two, more, or all of the fixation sections (BA2′, BA3′) are formed on island sections (IA).
